(19) United States
(12) Patent Application Publication
(10) Pub. No.: US 2023/0355845 A1
(43) Pub. Date: Nov. 9, 2023

(54) MEDICAL BALLOON WITH CELLULOSE-BASED FIBERS AND RELATED METHODS

(71) Applicant: C.R. BARD, INC., Franklin Lakes, NJ (US)

(72) Inventors: Matt Casiraro, Tempe, AZ (US); Rahim Jindani, Phoenix, AZ (US)

(21) Appl. No.: 18/027,272

(22) PCT Filed: Sep. 23, 2021
(86) PCT No.: PCT/US2021/051640

§ 371 Date: Mar. 20, 2023

Related U.S. Application Data
(60) Provisional application No. 63/082,077, filed on Sep. 23, 2020.

(51) Int. Cl. A61L 29/08; A61M 25/10

(52) U.S. Cl. CPC A61L 29/085; A61M 25/1029; A61L 2400/12; A61M 2025/1031; A61M 2025/1084

(57) ABSTRACT

A medical balloon includes a base balloon layer and at least one cellulose fiber applied to the base balloon layer, such as by an adhesive. The cellulose fiber may include hydro-dynamically focused, cellulose nano fibers. The cellulose fiber may be a longitudinal fiber extending along the base balloon substantially parallel to the longitudinal axis, at least one hoop fiber over the at least one longitudinal fiber, or both, including possibly as a single continuous fiber. The at least one fiber may further include silk proteins, and at least one silk fiber may also be included. An outer layer, such as a polymer film or spray coating, may be applied over the at least one cellulose fiber. Related methods are also disclosed.

This application claims the benefit of U.S. Provisional Patent Application Ser. No. 63/082,077, the disclosure of which is incorporated by reference.

### BACKGROUND

Medical balloons are widely used in medical procedures. Typically, an uninflated medical balloon is inserted into a body space. When the medical balloon is inflated, the volume of the medical balloon expands, and the body space is similarly expanded.

In procedures such as angioplasty, the medical balloon may be used to open a collapsed or blocked artery. A typical medical balloon includes a central barrel portion between tapered or conical portions. The medical balloon may be provided in a non-compliant form by including one or more fiber layers.

In the past, the fibers used in such balloons have typically been made of synthetic materials, such as UHMW polyethylene fiber. However, such synthetic fibers are not found in nature, and thus are costly to manufacture. Synthetic fibers may also lack superior adhesion characteristics in some applications.

Accordingly, a need is identified for a medical balloon that overcomes any or all of the foregoing limitations and possibly others that have yet to be identified.

### SUMMARY

According to a first aspect of the disclosure, a medical balloon includes a base balloon layer and at least one cellulose fiber applied to the base balloon layer.

In one embodiment, the base balloon layer includes first and second tapered portions and a barrel portion therebetween. The at least one cellulose fiber may extend over the first and second tapered portions and the barrel portion of the base balloon layer. The at least one cellulose fiber may extend substantially parallel to a longitudinal axis of the base balloon layer, and/or may extend radially around the base balloon layer. For example, in one particular embodiment, the at least one cellulose fiber comprises a single continuous fiber extending substantially parallel to a longitudinal axis of the base balloon layer and radially around the base balloon layer.

The medical balloon may further include at least one silk fiber. The at least one cellulose fiber may further comprise silk proteins. The at least one cellulose fiber may comprise cellulose nano fibers, such as hydro-dynamically focused cellulose nano fibers.

The at least one cellulose fiber may be attached to the base balloon layer by an adhesive. The at least one cellulose fiber may be attached to an outer surface of the base balloon layer. An outer layer may also be applied over the at least one cellulose fiber.

According to a further aspect of the disclosure, a medical balloon comprises a base balloon having a longitudinal axis and at least one longitudinal fiber extending along the base balloon substantially parallel to the longitudinal axis, the at least one longitudinal fiber comprising cellulose.

In one embodiment, including at least one hoop fiber extends over the at least one longitudinal fiber, the at least one hoop fiber comprising cellulose. The at least one longitudinal fiber and at least one hoop fiber may form part of a single continuous fiber. The at least one longitudinal fiber may further comprise silk proteins, and the medical balloon may further include at least one silk fiber. The at least one longitudinal fiber may comprise cellulose nano fibers, such as hydro-dynamically focused cellulose nano fibers. The at least one longitudinal fiber may be attached to the base balloon by an adhesive.

A further aspect of the disclosure pertains to a method of forming a medical balloon. The method comprises the step of applying at least one cellulose fiber applied to a base balloon layer.

In one embodiment, the applying step comprises applying the at least one cellulose fiber along a longitudinal axis of the base balloon. The applying step may comprise wrapping the cellulose fiber around the base balloon. The applying step may comprise adhesively attaching the at least one cellulose fiber to the base balloon.

### BRIEF DESCRIPTION OF THE DRAWINGS

The above and further advantages of the disclosure may be better understood by referring to the following description in conjunction with the accompanying drawings in which:

FIG. 1 illustrates a partial cross section of a medical balloon;

FIG. 2 illustrates an inflated balloon base layer;

FIG. 3 illustrates a balloon-shaped mandrel;

FIG. 4 illustrates a balloon base layer having an adhesive layer;

FIG. 5 illustrates a first fiber layer;

FIG. 6 illustrates a cross-section of a balloon base layer, adhesive layer and first fiber layer;

FIG. 7 illustrates a cross-section of a balloon base layer, adhesive layer and fiber layers;

FIG. 8 illustrates a cross-section of a balloon base layer, an adhesive layer, a first fiber layer, a second fiber layer, an outer coating layer and a final layer;

FIG. 9 illustrates a fiber-reinforced medical balloon with a longitudinal first fiber layer and a circumferential second fiber layer;

FIG. 10 illustrates a fiber-reinforced medical balloon with a first angled fiber layer and a second longitudinal second fiber layer;

FIG. 11 illustrates a fiber-reinforced medical balloon having a first longitudinal fiber layer and a second angled fiber layer;

FIG. 12 illustrates a fiber-reinforced medical balloon having a longitudinal first fiber layer and an angled second fiber layer;

FIG. 13 illustrates an alternative embodiment of a fiber-reinforced medical balloon; and

FIGS. 14, 15, 15A, and 16 illustrate a fiber-reinforced medical balloon forming part of a catheter.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present invention.

The principles and operation of systems and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

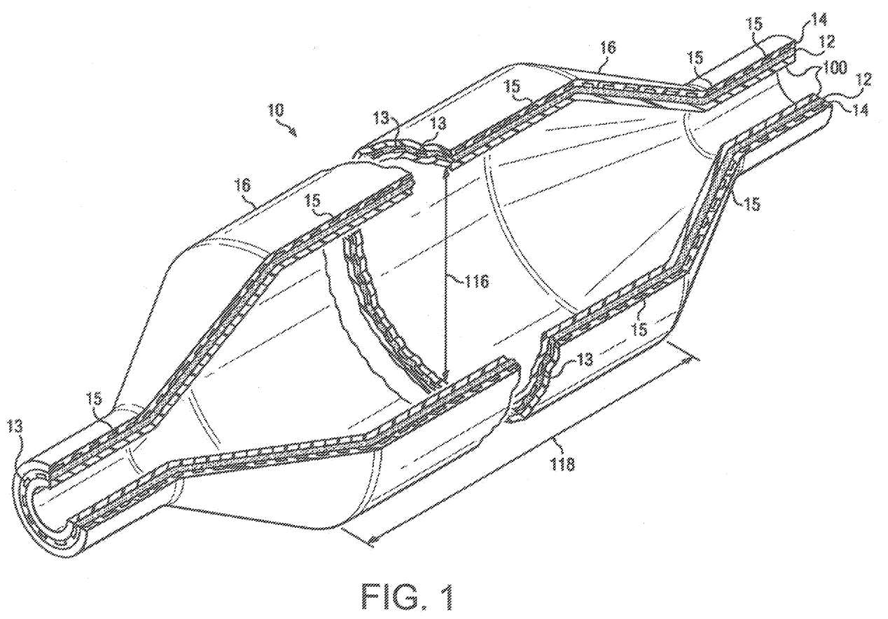

With reference to FIG. 1, a partial cross section of an inflated composite medical balloon 10 is shown. In the illustrated embodiment, and as discussed further in the following description, the balloon 10 may optionally be fiber-reinforced and, as a result, is substantially non-compliant, having limited expansion characteristics. Because the medical balloon 10 is non-compliant, once fully inflated, a diameter 116 of it does not substantially change as the interior pressure increases. As noted further below, the use of a fiber layer is considered entirely optional.

The diameter 116 of an inflated fiber-reinforced medical balloon 10 in accordance with the one embodiment may be about ten millimeters, but may vary depending on the application. The length of an inflated fiber-reinforced medical balloon 10 in accordance with one embodiment may be about eight centimeters. A balloon 10 with a length 118 of 2-200 centimeters or more is possible. The inclination angle of a tapered or cone portion 108 of an inflated fiber-reinforced medical balloon 10 in accordance with the disclosed embodiment may be about twenty degrees.

It will be recognized by those having skill in the art that the fiber-reinforced balloon 10 could be made in a wide variety of diameters 116 and lengths and with a variety of inclinations at the tapered or cone portion 108 of the balloon 10, without limitation.

The fiber-reinforced medical balloon 10 may include a base layer 100 formed of a thin polymer material and a first layer 12 of thin, elongated inelastic fibers 13, which may extend longitudinally. The balloon 10 may include a second layer 14 made up of one or more thin, elongated fibers 15, which may extend transversely (termed “hoop” fibers). Additional fiber layers may be included, if desired.

An outer layer 16 over the fiber layer(s) 12, 14 (or others, if present) may be included. This outer layer 16 may comprise a polymer. The polymer may be applied in film form (blow-molded, extruded, or otherwise) or applied as a solution, such as by spraying.

Each fiber 13 is typically fixed relative to other fibers in the first fiber layer 12 and other fibers in the balloon 10. The thin inelastic fibers 13 of the first fiber layer 12 may be characterized by a high tensile strength, which provide superior burst strength. The fiber-reinforced balloon 10 may also resist abrasion, cuts, and punctures. It may be recognized that enhanced structural integrity may result from the fiber reinforcement.

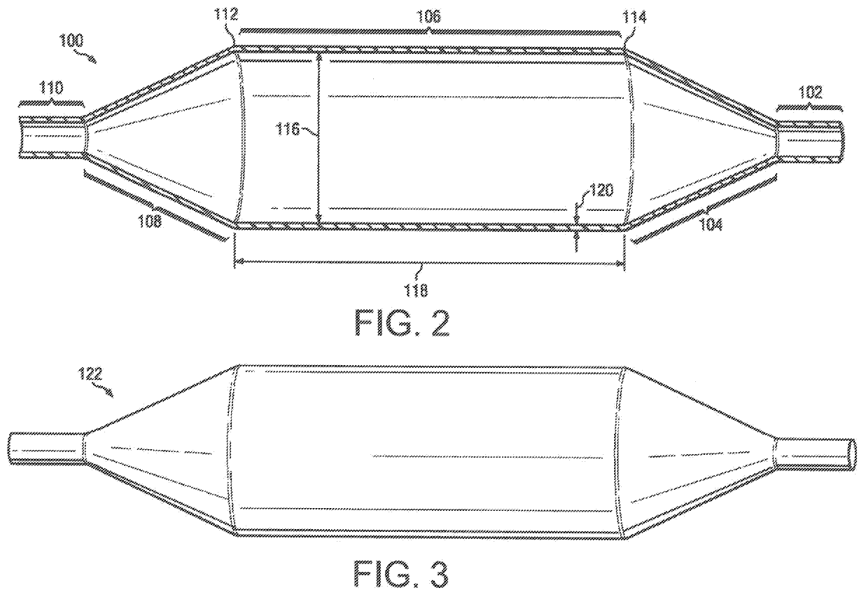

With further reference to FIG. 2, the base layer 100 may be in the shape of a standard medical balloon, or any other suitable shape. The base layer 100 typically includes a first neck portion 102 that may be formed as a narrow cylinder fashioned to attach to a catheter shaft (see FIGS. 19, 20 and 21). A second neck portion 110 may be similarly formed as a narrow tube. The first neck portion 102 is formed adjacent to a first cone or tapered portion 104. The first cone portion 104 expands to meet a barrel portion 106 having a working length 118, marked by a first transition 114. The first cone portion 104 is typically constructed at an angle of about twelve to twenty degrees. The central region or barrel portion 106 meets the second cone or tapered portion 108 at a second transition 112. The second cone portion 108 meets the second neck portion 110.

The base layer 100 is typically formed of a thin film polymeric material, or other suitable materials with high strength relative to film thickness. Polymers and copolymers that can be used for the base layer 100 include the conventional polymers and copolymers used in medical balloon construction, such as, but not limited to, polyethylene, (PET), polycaprolactam, polyesters, polyethers, polyamides, polyurethanes, polyimides, ABS, nylons, copolymers, polyester/polyether block copolymers, ionomer resins, liquid crystal polymers, and rigid rod polymers. The base layer 100 may typically be formed as a blow-molded balloon of a polymer material, such as for example a polyamide, such as nylon.

The base layer 100 may comprise a polymer, which has been cured into the shape of a balloon, may be formed. This base layer 100 of a cured polymer may form the inner polymeric wall of the fiber reinforced balloon. With reference to FIG. 3, a removable mandrel 122 may be used as a base for application of the polymer coating. After the polymer is cured, the mandrel 122 may be removed, such as by physical withdrawal, heat, or other known forms of dissolution.

A removable balloon similar to base layer 100 may be used as the mandrel 122. The mandrel 122 may be made from a variety of materials. The mandrel 122 may be made in the shape of the interior wall of the desired finished balloon. The mandrel 122 may be made of collapsible metal or polymeric bladder, foams, waxes, low-melting metal alloys, and the like. Once the composite balloon is developed and laminated, the mandrel 122 (i.e., base layer 100) may be removed by melting, dissolving, fracturing, compressing, pressurizing, or other suitable removal techniques.

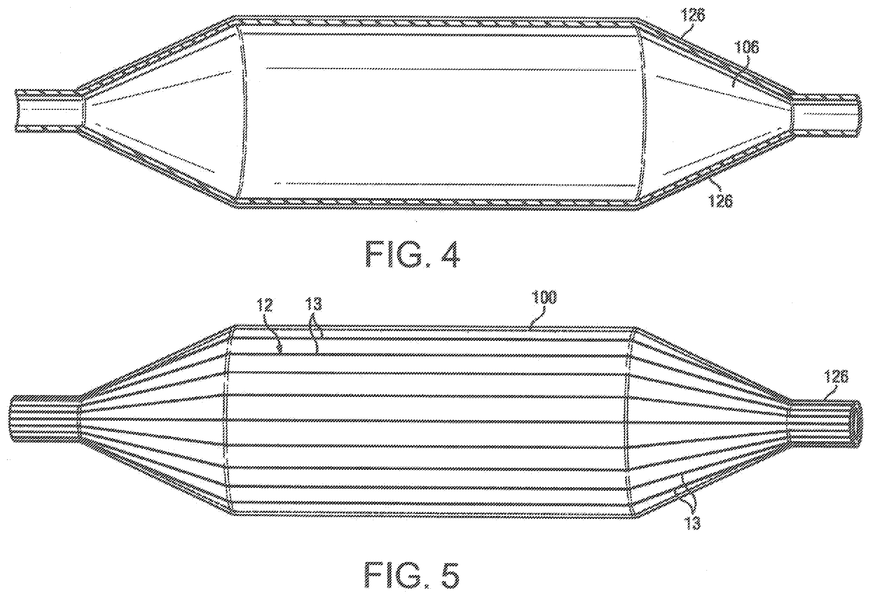

With reference to FIG. 4, it can be understood that a thin coating of an adhesive 126 is applied to the inflated base layer 100 or to the polymer-coated mandrel 122 prior to applying the first layer inelastic fibers 12. The adhesive 126 binds the fibers 13 sufficiently to hold them in position when the fibers 13 are placed on the base layer 100. In accordance with one embodiment, a very thin coat of adhesive 126 is applied to the base layer 100, such as for example 1-MP adhesive, which is a known solution of a polyurethane based polymer and methyl ethyl ketone and methylene chloride, but other forms of adhesive could be used.

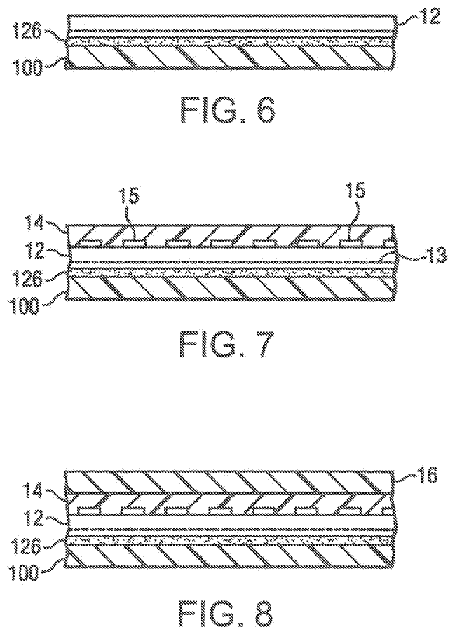

One or more fibers 13 are applied to the base layer 100 to form a first fiber layer 12, as shown in FIGS. 5 and 6. The fibers 13 of the first fiber layer 12 may be inelastic fiber. An inelastic fiber is a fiber that has very minimal elasticity or stretch over a given range of pressures. Some fibrous materials are generally classified as inelastic although all fibrous material may have a detectable, but minimal, elasticity or stretch at a given pressure.

In a disclosed embodiment, the fibers 13 of the first fiber layer 12 are ribbon-shaped, where the width of the fiber is larger than the thickness of the fiber. The fibers 13 may be flat so that the fiber has a rectangular cross-section. The fibers 13 used in the initial layer of fibers 12 may all be fibers 13 made of the same material and the same shape. Fibers 13 made from different materials may be used in the initial fiber layer 12. Fibers 13 made in different shapes may be used in the initial fiber layer 12. Fiber 13, which has been flattened on a roll mill may be used to form the first fiber layer 12. To the flattened fiber 13 is applied an adhesive, such as the 1-MP adhesive. The fibers 13 may be arranged as 30 longitudinal fibers, each substantially equal in length to the length of the balloon 10.

The fibers 13 of the initial fiber layer 12 may be arranged so that each fiber 13 is substantially parallel to the long axis of the balloon 10. The density of the fibers 13 in the initial fiber layer 12 is determined by the number of fibers 13 or fiber winds per inch and the thickness of the fibers 13. In a disclosed embodiment of the first fiber layer 12 having longitudinally-placed fibers 13, a fiber density of generally about 15 to 30 fibers 13 having a fiber thickness of about 0.0005 to 0.001 inch and placed equidistant from one another provide adequate strength for a standard-sized fiber-reinforced medical balloon 10. Each of the fibers 13 is substantially equal in length to the balloon 10. The first fiber layer 12 may prevent longitudinal extension of the completed fiber-reinforced balloon 10.

In accordance with a disclosed embodiment, a second fiber layer 14 made with one or more high-strength inelastic fibers 15 is positioned along circumference of the balloon 10, as shown in FIG. 7. The circumferentially placed fibers 15 may be transverse or substantially transverse to the longitudinal axis of the balloon 10. The circumferential fibers 15 may prevent or minimize distension of the balloon diameter 116 at pressures between the minimal inflation pressure and the balloon burst pressure.

The fibers 15 of the second fiber layer 14 may be inelastic fiber, typically made of an inelastic fibrous material. An inelastic fiber is a member of a group of fibers that have very minimal elasticity or stretch in a given range of pressures. Some fibrous materials are generally classified as inelastic although the all fibrous material may have a detectable, but minimal elasticity or stretch at a given pressure. The fibers 15 of the second fiber layer 14 may be high-strength fibers, typically made of a high-strength fibrous material.

In a disclosed embodiment, the fibers 15 of the second fiber layer 14 are ribbon-shaped, where the width of the fiber is larger than the thickness of the fiber. The fibers 15 may be flat so that the fiber has a rectangular cross-section. The fibers 15 used in the second layer of fibers 14 may all be fibers 15 made of the same material and the same shape. Fibers 15 made from different materials may be used in the second fiber layer 14. Fibers 15 made in different shapes may be used in the second fiber layer 14. Fiber 15 which has been flattened on a roll mill may be used to form the second fiber layer 14. To the flattened fiber 15 is applied a thin coat of an adhesive, such as the 1-MP adhesive. The fibers 15 may be arranged as a second fiber layer 14 may have a fiber density of 54 wraps per inch.

The fibers 15 of the second fiber layer 14 may be perpendicular to or substantially perpendicular to the fibers 13 placed longitudinally to form the first fiber layer 12. This transverse placement of the first fiber layer 12 and the second fiber layer 14 allows for maximum radial stability of the fiber-reinforced balloon 10. The placement of the fiber layers 12, 14 distributes the force on the balloon surface equally, creating pixelized pressure points of generally equal shape, size, and density.

The fibers 13 of the first fiber layer 12 may be the same as or different from the fiber 15 of the second fiber layer 14. Specifically, the fibers 15 of the second fiber layer 14 may be made of a different material or materials than the fibers 13 of the first layer 12. The fibers 15 of the second layer 14 may be shaped differently from the fibers 13 of the first fiber layer 12. The characteristics of the fibers or combination of fibers used for the first or second fiber layers may be determined from the specific properties required from the resulting fiber-reinforced balloon 10.

With respect to the fiber density of the second fiber layer 14, in accordance with the disclosed embodiment, fiber 15 having a thickness of about 0.0005 to 0.001 inch and arranged in parallel lines with about 50 to 80 wraps per inch provides generally adequate strength. A single fiber 15 may form the second fiber layer 14, with the fiber 15 wound in a generally parallel series of circumferential continuous loops.

With reference to FIG. 8, a cross section of the integral layers of a fiber-reinforced medical balloon 10 is shown. The first fiber layer 12 and the second fiber layer 14 may be coated with an outer layer 16. The outer layer 16 may be, in the disclosed embodiment, a hybrid outer layer, as outlined further in the following description. A composite structure typically including a base layer 100, an adhesive 126, a first fiber layer 12, a second fiber layer 14 and an outer layer 16 forming a composite, non-compliant fiber-reinforced balloon 10 particularly suitable for medical uses. Typically, the fibers 13 and 15 are fixed when the fiber-reinforced balloon 10 is initially deflated, and then subsequently inflated and deflated during use.

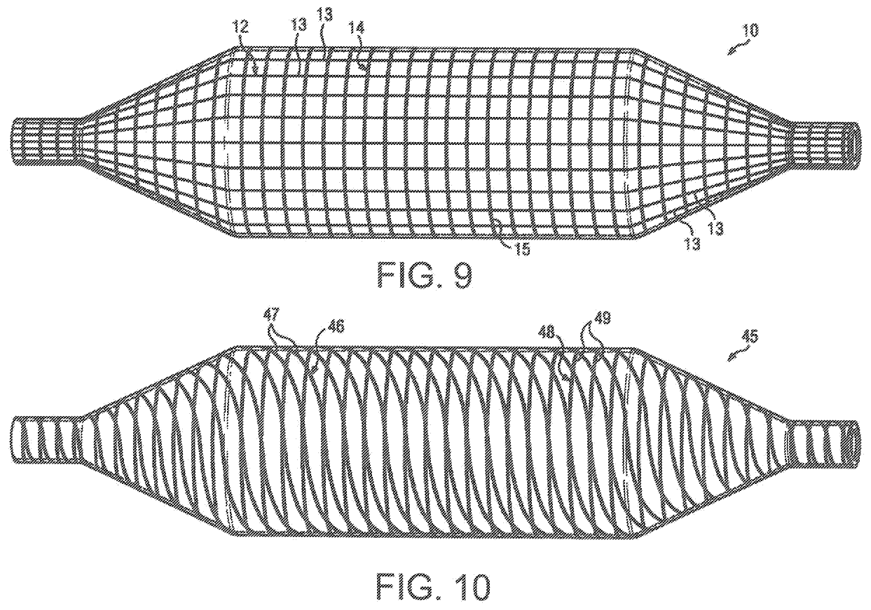

With reference to FIG. 9, a fiber reinforced balloon 10 in accordance with the disclosed embodiment, is shown. In this embodiment, the fibers 13 are parallel to the balloon 10 long axis.

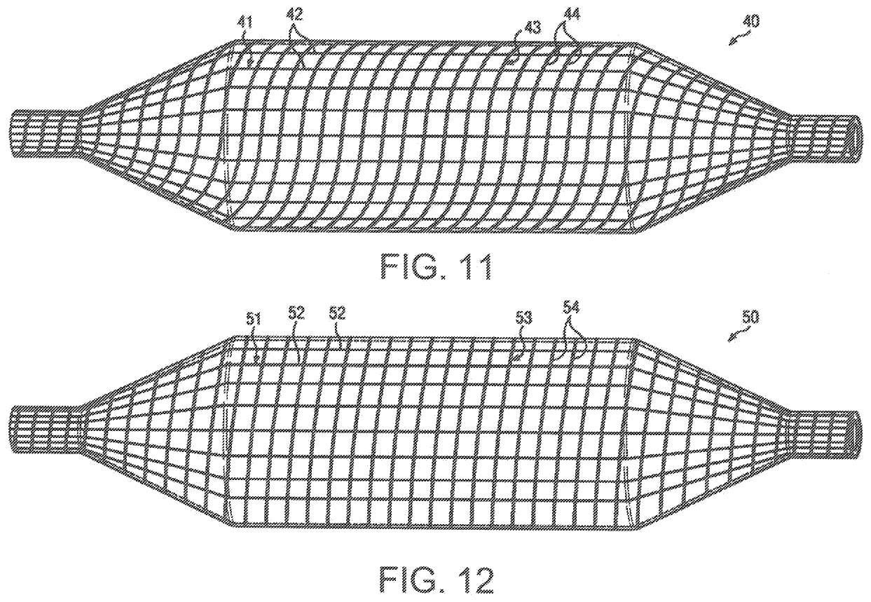

With reference to FIG. 10, a fiber reinforced balloon 45, in accordance with another embodiment is shown. The fiber-reinforced balloon 45 may include a first fiber layer 46 with fibers 47 that lie at an angle to the longitudinal axis of the balloon 45. In this embodiment, neither the fibers 47 of the first fiber layer 46 nor the fibers 49 of the second fiber layer 48 are positioned parallel to the longitudinal axis of the balloon 45. In accordance with one embodiment, the fibers 47 of the first fiber layer 46 may be positioned parallel to a line at a five-degree angle to a line parallel to the longitudinal axis of the balloon 10. In accordance with another embodiment, the fibers 47 of the first fiber layer 46 may be positioned parallel to a line at a twenty-degree angle to a line parallel to the longitudinal axis of the balloon 10.

In accordance with another embodiment, the fibers 47 of the first fiber layer 46 may be positioned parallel to a line at a thirty-degree angle to a line parallel to the longitudinal axis of the balloon 10. In accordance with another embodiment, the fibers 47 of the first fiber layer 46 may be positioned parallel to a line at a forty-five-degree angle to a line parallel to the longitudinal axis of the balloon 10. It will be apparent to those having skill in the art that the fibers 47 may be placed at any appropriate angle.

The fibers 49 of the second fiber layer 48 lie parallel to the circumference of the balloon 10. With reference to FIG. 11, a fiber-reinforced balloon 40 in accordance with another embodiment is shown. The fiber reinforced balloon 40 may include a second fiber layer 43 with fibers 44 that lie at an angle to the circumference of the balloon 40. In accordance with one embodiment, the fibers 44 of the second fiber layer 43 may be positioned parallel to a line at a five-degree angle to a line parallel to the circumference of the balloon 10.

The fiber 44 of the second fiber layer 43 may be positioned parallel to a line at a twenty-degree angle to a line parallel to the circumference of the balloon 10. In accordance with one embodiment, the fiber 44 of the second fiber layer 43 may be positioned parallel to a line at a thirty-degree angle to a line parallel to the circumference of the balloon 10. In accordance with one embodiment, the fiber 44 of the second fiber layer 43 may be positioned parallel to a line at a forty-five-degree angle to a line parallel to the circumference of the balloon 10. It will be apparent to those skilled in the art that the fibers 44 may be placed at any appropriate angle.

The fibers 42 of the first fiber layer 41 and the fibers 44 of the second fiber layer 43 are positioned perpendicularly relative to each other. With reference to FIG. 12, a fiber-reinforced balloon 50 in accordance with another embodiment is shown. A fiber-reinforced balloon 50 may include fibers 52 of the first fiber layer 51 and fibers 54 of the second fiber layer 53 positioned relatively at an angle other than a right angle.

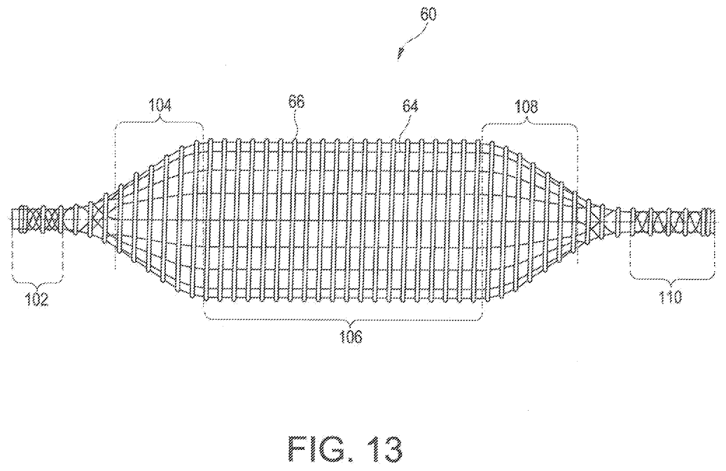

Referring to FIG. 13, a medical balloon 60 may also include a single continuous fiber forming a longitudinal fiber strand 64 and a hoop fiber strand 66. Specifically, the fiber 62 may be wrapped circumferentially around one end portion, such as neck portion 102, and directed longitudinally to the other end portion, or neck portion 110 (possibly at a non-zero angle to the longitudinal axis). The fiber 62 is then wrapped around the other end portion 110, and returned in the opposite longitudinal direction (again, possibly at a non-zero angle). The single continuous fiber 62 can continue to be wound back and forth along the balloon 60 for a desired number of longitudinal passes, and then would circumferentially or helically around the balloon 60 at a desired fiber pitch. A more complete description of the application to a single continuous fiber to a medical balloon 60 may be found in U.S. Pat. No. 10,485,949.

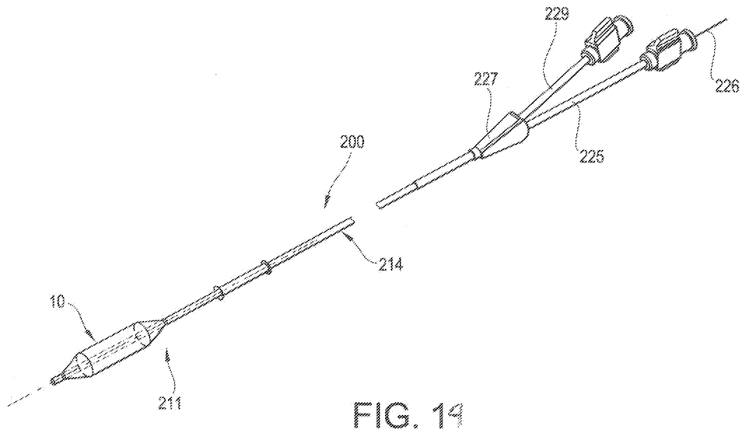

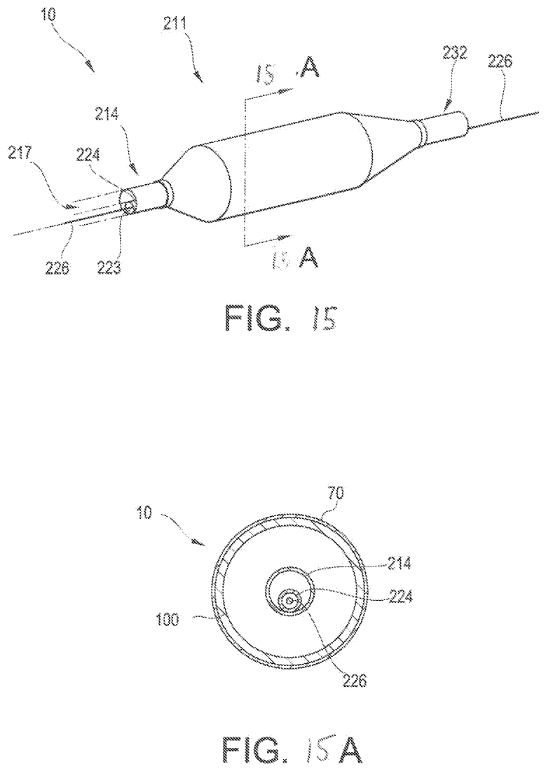

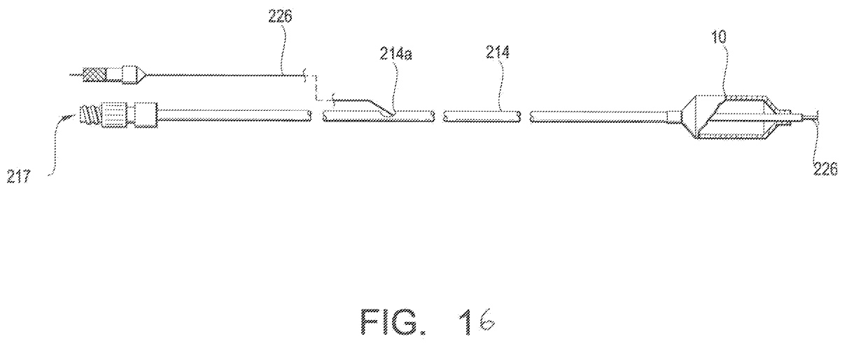

Turning to FIGS. 14-15, it can be understood that the balloon 10 may form part of a catheter 200 having a shaft 214 with a distal end portion 211 to which the balloon 10 is mounted. The balloon 10 is sealed at balloon ends to allow the inflation via one or more inflation lumens 217 extending within catheter shaft 214 and communicating with the interior of the balloon 10. The catheter 200 may also include a guidewire lumen 223 formed by a shaft 224, which may be within the shaft 214 and, more particularly, within the inflation lumen 217. This lumen 223 directs the guidewire 226 through the catheter 200 (see FIG. 15A), and along the distal end portion of which the balloon 10 may be located, including through a tip 232 distal of balloon 10 distal end.

As illustrated in FIG. 15, this guidewire 226 may extend through the proximal end portion of the catheter 200 via a first port 225 of a connector or hub 227 at a proximal end portion 213 of the shaft 214 into the lumen 223 to achieve an “over the wire” (OTW) arrangement, but could also be provided in a “rapid exchange” (RX) configuration (in which the guidewire 226 exits from the shaft 214 at an optional lateral opening 214a (see in FIG. 16) closer to the distal end but proximal of balloon 10) or else is fed through the tip 232 at a passage distally of the balloon 10 (“short” RX; not shown). A second port 229 may also be associated with catheter 200, such as by way of connector 227, for introducing a fluid (e.g., saline, a contrast agent, or both) into the interior compartment of the balloon 10 via the inflation lumen 217.

The fibers 13 of the first fiber layer 12 and/or the fibers 14 of the second fiber layer 15, (or any other fibers disclosed herein, such as the fibers 47 of the first fiber layer 46 and fibers 49 of the second fiber layer 48) may comprise natural materials, such as cellulose. Specifically, any or all of these fibers may comprise one or more of: (1) cellulose nano fibers (fibrils); (2) cellulose nano fibers combined with silk fibers; or (3) cellulose nano fibers combined with silk proteins. Advantageously, the fibers 13 or 15 may thus comprise naturally available, as compared to synthetic, materials, which can reduce the cost of manufacturing or use without sacrificing strength or crimpability.

The cellulose-based fibers or fibrils may be formed using a process called hydrodynamic focusing. This involves forming a fiber from cellulose nano fibers by feeding them into a water channel that squeezes/condenses the fibers into an aligned, well packed and extremely strong fiber material. Such fibers may have stiffness of 60-70 Gpa with tensile break of 1015 MPa, and have superior properties of adhesion as compared to synthetic fibers.

Summarizing, this disclosure may relate to the following items:

1. A medical balloon comprising:
a base balloon layer; and
at least one cellulose fiber applied to the base balloon layer.

2. The medical balloon of item 1, wherein the base balloon layer includes first and second tapered portions and a barrel portion therebetween.

3. The medical balloon of item 1 or item 2, wherein the at least one cellulose fiber extends over the first and second tapered portions and the barrel portion of the base balloon layer.

4. The medical balloon of any of items 1-3, wherein the at least one cellulose fiber extends substantially parallel to a longitudinal axis of the base balloon layer.

5. The medical balloon of any of items 1-4, wherein the at least one cellulose fiber extends radially around the base balloon layer.

6. The medical balloon of item 1, wherein the at least one cellulose fiber comprises a single continuous fiber extending substantially parallel to a longitudinal axis of the base balloon layer and radially around the base balloon layer.

7. The medical balloon of any of items 1-6, further including at least one silk fiber.

8. The medical balloon of any of items 1-7, wherein the at least one cellulose fiber further comprises silk proteins.

9. The medical balloon of any of items 1-8, wherein the at least one cellulose fiber comprises cellulose nano fibers.

10. The medical balloon of any of items 1-8, wherein the at least one cellulose fiber comprises hydrodynamically focused cellulose nano fibers.

11. The medical balloon of any of items 1-8, wherein the at least one cellulose fiber is attached to the base balloon layer by an adhesive.

12. The medical balloon of any of items 1-8, wherein the at least one cellulose fiber is attached to an outer surface of the base balloon layer.

13. The medical balloon of any of items 1-12, wherein an outer layer, such as a polymer film or spray coating, is applied over the at least one cellulose fiber.

14. A medical balloon comprising:
a base balloon having a longitudinal axis; and
at least one longitudinal fiber extending along the base balloon substantially parallel to the longitudinal axis, the at least one longitudinal fiber comprising cellulose.

15. The medical balloon of item 14, further including at least one hoop fiber over the at least one longitudinal fiber, the at least one hoop fiber comprising cellulose.

16. The medical balloon of item 15, wherein the at least one longitudinal fiber and at least one hoop fiber form part of a single continuous fiber.

17. The medical balloon of any of items 14-16, wherein the at least one longitudinal fiber further comprises silk proteins.

18. The medical balloon of any of items 14-17, further including at least one silk fiber.

19. The medical balloon of any of items 14-18, wherein the at least one longitudinal fiber comprises cellulose nano fibers.

20. The medical balloon of any of items 14-19, wherein the at least one longitudinal fiber is formed of hydro-dynamically focused cellulose nano fibers.

21. The medical balloon of any of items 14-20, wherein the at least one longitudinal fiber is attached to the base balloon by an adhesive.

22. A method of forming a medical balloon, comprising:
applying at least one cellulose fiber applied to a base balloon layer.

23. The method of item 22, wherein the applying step comprises applying the at least one cellulose fiber along a longitudinal axis of the base balloon.

24. The method of item 22 or item 23, wherein the applying step comprises wrapping the cellulose fiber around the base balloon.

25. The method of any of items 22-24, wherein the applying step comprises adhesively attaching the at least one cellulose fiber to the base balloon.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the appended claims' spirit and scope. All publications, patents and patent applications mentioned in this specification are herein incorporated in their entirety by reference into the specification, to the same extent as if each individual publication, patent or patent application was specifically and individually indicated to be incorporated herein by reference. In addition, the identification of any reference in this application shall not be construed as an admission that such reference is available as prior art to the present disclosure.

1. A medical balloon comprising:
a base balloon layer; and
at least one cellulose fiber applied to the base balloon layer.

2. The medical balloon of claim 1, wherein the base balloon layer includes first and second tapered portions and a barrel portion therebetween.

3. The medical balloon of claim 1, wherein the at least one cellulose fiber extends over the first and second tapered portions and the barrel portion of the base balloon layer.

4. The medical balloon of claim 1, wherein the at least one cellulose fiber extends substantially parallel to a longitudinal axis of the base balloon layer.

5. The medical balloon of claim 1, wherein the at least one cellulose fiber extends radially around the base balloon layer.

6. The medical balloon of claim 1, wherein the at least one cellulose fiber comprises a single continuous fiber extending substantially parallel to a longitudinal axis of the base balloon layer and radially around the base balloon layer.

7. The medical balloon of claim 1, further including at least one silk fiber.

8. The medical balloon of claim 1, wherein the at least one cellulose fiber further comprises silk proteins.

9. The medical balloon of claim 1, wherein the at least one cellulose fiber comprises cellulose nano fibers.

10. The medical balloon of claim 1, wherein the at least one cellulose fiber comprises hydro-dynamically focused cellulose nano fibers.

11. The medical balloon of claim 1, wherein the at least one cellulose fiber is attached to the base balloon layer by an adhesive.

12. The medical balloon of claim 1, wherein the at least one cellulose fiber is attached to an outer surface of the base balloon layer.

13. The medical balloon of claim 1, wherein an outer layer is applied over the at least one cellulose fiber.

14. A medical balloon comprising:
a base balloon having a longitudinal axis; and
at least one longitudinal fiber extending along the base balloon substantially parallel to the longitudinal axis, the at least one longitudinal fiber comprising cellulose.

15. The medical balloon of claim 14, further including at least one hoop fiber over the at least one longitudinal fiber, the at least one hoop fiber comprising cellulose.

16. The medical balloon of claim 15, wherein the at least one longitudinal fiber and at least one hoop fiber form part of a single continuous fiber.

17. The medical balloon of claim 14, wherein the at least one longitudinal fiber further comprises silk proteins.

18. The medical balloon of claim 14, further including at least one silk fiber.

19. The medical balloon of claim 14, wherein the at least one longitudinal fiber comprises cellulose nano fibers.

20. The medical balloon of claim 14, wherein the at least one longitudinal fiber is formed of hydro-dynamically focused cellulose nano fibers.

21. The medical balloon of claim 14, wherein the at least one longitudinal fiber is attached to the base balloon by an adhesive.

22. A method of forming a medical balloon, comprising:
applying at least one cellulose fiber applied to a base balloon layer.

23. The method of claim 22, wherein the applying step comprises applying the at least one cellulose fiber along a longitudinal axis of the base balloon.

24. The method of claim 22, wherein the applying step comprises wrapping the cellulose fiber around the base balloon.

25. The method of claim 22, wherein the applying step comprises adhesively attaching the at least one cellulose fiber to the base balloon.
